(19) United States
(12) Patent Application Publication
(10) Pub. No.: US 2018/0201982 A1
(43) Pub. Date: Jul. 19, 2018

(54) DISPOSABLE SENSOR

(71) Applicant: THE UNIVERSITY COURT OF THE UNIVERSITY OF EDINBURGH, EDINBURGH MIDLOTHIAN (GB)

(72) Inventors: MICHAEL P. SHAVER, EDINBURGH (GB); PHILLIP J.W. HANDS, EDINBURGH (GB); DAVID FERRIER, EDINBURGH (GB)

(21) Appl. No.: 15/742,663

(22) PCT Filed: Jul. 7, 2016
(86) PCT No.: PCT/GB2016/052045

§ 371 Date: Jan. 8, 2018

(30) Foreign Application Priority Data
Jul. 8, 2015 (GB) 1511969.6

(51) Int. Cl. C12Q 1/6825; G01N 27/327

(52) U.S. Cl. CPC C12Q 1/6825; C12Q 2525/205; G01N 27/3276

(57) ABSTRACT

A biosensor comprising a swellable biologically sensitive element comprising a nucleic acid probe and a physicoelectrical transducer associated with the swellable biologically sensitive element. The physicoelectrical transducer converts the swelling of the swellable biologically sensitive element into a detectable electrical property. In preferred embodiments the biosensor comprises an oligonucleotide cross-linked polymer composite mounted on a suitable substrate comprising electrodes adapted to measure electrical impedance. Associated methods and devices comprising said biosensor are also provided.

The present invention relates to biosensors and systems comprising biosensors. In some embodiments the biosensors are adapted to detect nucleic acids in a sample.

Biosensors are an increasingly important set of analytical tools for use in various technical areas, not least in diagnosis. In many cases they combine a biological recognition (bio-recognition) component and a suitable transducer. They can generally convert a biochemical signal into a suitable signal in the presence of an analyte which interacts with the bio-recognition component.

Biosensors for the detection of nucleic acids are of particular importance. In view of the ongoing development of the use of micro RNAs (miRNAs) as biomarkers for diagnosis and other biological investigations, the need for sensors which rapidly and conveniently identify nucleic acids in a sample has increased. Biosensors for nucleic acids can make use of the interaction between two complementary nucleic acid strands to activate the bio-recognition component of the biosensor. Transduction of this interaction into a measurable electrical signal is challenging.

While there are various known methods for detecting nucleic acids (e.g. miRNAs) in a sample, there remains a pressing need for further biosensors.

Speaking more broadly, there is also a need for novel biosensors for other types of target.

In particular, biosensors with lower costs, improved convenience, faster detection speeds and/or improved sensitivity than known biosensor platforms are desirable.

In accordance with a first aspect of the present invention there is provided a biosensor comprising:
a swellable biologically sensitive element comprising a nucleic acid probe; and
a physicoelectrical transducer associated with the swellable biologically sensitive element.

The swellable biologically sensitive element comprising the nucleic acid probe is adapted for the recognition of a target/analyte.

In certain embodiments of the invention the target can be a nucleic acid. In this case target nucleic acids can suitably be recognised by the nucleic acid probe through complementary base-pairing (hybridisation) between the nucleic acid probe and target nucleic acid.

In other embodiments the target can be a non-nucleic acid target, which can be any suitable target entity, for example, a protein/peptide, drug, lipid, polysaccharide, other small molecules, cell surface, etc. In these embodiments the nucleic acid probe is preferably a nucleic acid aptamer. Aptamers are a well-described class of nucleic acid-based moieties which can bind, often with high specificity and affinity, to a wide range of targets ranging from macromolecules and larger structures to small compounds. Nucleic acid aptamers can be DNA, RNA or non-natural nucleic acids, which can be further modified in various ways. The Aptamer Database (http://aptamer.icmb.utexas.edu/) has been established which catalogues substantially all published experiments using aptamers. Accordingly, in some preferred embodiments the nucleic acid probe comprises an aptamer.

Where the target is a nucleic acid, it can be essentially any nucleic acid, but in many cases it is an oligonucleotide, such as a miRNA. Preferably the target nucleic acid is single stranded when it is exposed to the biosensor as this facilitates hybridisation with the nucleic acid probe. In the case of miRNAs, the single stranded form is the normal state. Alternatively, single stranded forms can be generated to facilitate interaction with the nucleic acid probe. This can be done in situ in the biosensor, or before a sample is applied to the biosensor. For example, double stranded (duplex) DNA can be readily separated into single strands (melting or denaturing) by heating and/or administering a suitable chemical agent (e.g. urea); subsequent removal of the excess heat and/or chemical agent will allow at least a proportion of the target nucleic acids to anneal with the nucleic acid probe. However, in view of the dynamic nature of hybridisation of nucleic acids, the present invention can typically still function when the target nucleic acid is double stranded when it is exposed to the biosensor.

In use the biologically sensitive element allows for a physical effect, i.e. swelling of a polymer, when exposed to a sample comprising the appropriate target. This physical effect is the result of an interaction between the target and the nucleic acid probe. The physicoelectrical transducer then transforms the physical effect (physical signal) into a detectable/measurable electrical indicator (e.g. a measurable electrical property such as impedance or resistance or an electrical signal such as a voltage or current) that can be measured and/or monitored by a user. Preferably, the electrical property to be detected is a change in electrical impedance (or admittance, defined as the inverse of the impedance), and may therefore include changes in resistance, conductance, reactance, susceptance, capacitance, inductance, or combinations thereof. Other forms of detectable changes in electrical properties can alternatively be used, e.g. dielectric constant, electrical permittivity, electrical permeability.

Preferably the swellable biologically sensitive element is adapted such that binding of nucleic acid probe to a corresponding target results in an increase in the swellability of the swellable biologically sensitive element. For example, the nucleic acid probe can define part of a frangible (i.e. readily-cleavable) cross-linker in a swellable polymeric material, wherein the cross-linker is cleaved when the target binds to the nucleic acid probe.

In some embodiments the swellable biologically sensitive element suitably comprises at least one pair of at least partially complementary nucleic acid strands, at least one strand of each pair comprising a nucleic acid probe. Pairs of partially complementary nucleic acids are able to hybridise to form a frangible cross-linker, the frangible cross-linker being cleaved when the pair are separated.

Preferably the frangible cross-linker comprises at least one pair of partially complementary nucleic acids which comprise a nucleic acid probe strand and a blocker strand. The nucleic acid probe strand and blocker strand are at least partially complementary to one another, to allow them to anneal, but it is highly preferred that they are imperfectly complementary to one another and/or only perfectly complementary over a portion of their total lengths.

The nucleic acid probe strand and blocker strand are typically in a single stranded form until they anneal to one another when forming the frangible cross-linker (they are typically annealed prior to incorporation into a polymer), or when the nucleic acid probe binds to a target analyte (e.g. a complementary polynucleotide such as a miRNA).

In such embodiments the nucleic acid probe can be adapted to bind to a nucleic acid target through hybridisation, or it can be a nucleic acid aptamer adapted to bind to a non-nucleic acid target. It will be appreciated that a nucleic acid aptamer is capable of binding to a complementary strand, such as a blocker strand, in the same way as any other nucleic acid. In the presence of the aptamer target the target will compete with the blocker for binding to the aptamer, thus leading to cleavage of the cross-linker.

In alternative embodiments the swellable biologically sensitive element suitably comprises two nucleic acid blocker strands linked to the polymer and a nucleic acid probe sequence which is at least partially complementary to, and thus hybridises to, both of the blocker sequences to form a frangible crosslink. Typically, the nucleic acid probe sequence will have a first sequence (typically at or near one end) which is at least partially complementary to a first blocker, and a second sequence (typically at or near the other end) which is at least partially complementary to a second blocker. Thus, in such embodiments the nucleic acid probe forms a bridge between two nucleic acid blocker sequences anchored in the polymer to form a three-part cross-link. The nucleic acid probe in this case can again be an aptamer or a probe intended to bind to a target nucleic acid through base pairing.

In alternative embodiments the swellable biologically sensitive element suitably comprises a nucleic acid aptamer probe and a corresponding target for the aptamer. Thus the frangible cross-link is suitably formed by a nucleic acid aptamer and a non-nucleic acid target (which can be referred to as a blocking target). Integration of a suitable target into a swellable polymeric material can be achieved via many conventional chemical techniques, the appropriate technique will obviously be determined by the nature of the polymer and the target. Conveniently, when a target is integrated into a polymeric material there can be a reduction in the affinity of the aptamer for the target due to resultant slight changes in the target. Such a reduction in affinity confers the benefit that the aptamer will preferentially bind to the ‘natural’ target in a biological sample compared with the blocking target.

When an aptamer (or indeed any other sequence) is linked to a monomer/polymer it may be desirable to include a spacer between the polymer and the binding sequence, e.g. so that adoption of the correct conformation by the aptamer for binding the target is not prevented by, for example, steric hindrance or other effects arising the polymer. The spacer can be a nucleic acid spacer or it can be any other suitably spacer such as a hydrocarbon chain, e.g. a (CH₂)ₙ chain, where n can be any suitable number, preferably from 1 to 10. A nucleic acid spacer of any suitable length can be used, preferably from 1 to 20 nucleotides, more preferably from 2 to 10 nucleotides, for example about 5 nucleotides in length.

The frangible cross-linker may thus comprise one or more pairs of imperfectly complementary nucleic acids. ‘Imperfectly complementary’ in this case means that the matching of complementary bases between the two nucleic acid strands in the frangible cross-linker is lesser than that the matching of complementary bases between the nucleic acid probe with its complementary target (e.g. miRNA or other nucleic acid, or non-nucleic acid target). In other words, the binding of the nucleic acid probe to the target is stronger (and thus thermodynamically more favoured) than binding to the blocker strand. This means that in the presence of the target, binding of the nucleic acid probe to the blocker becomes thermodynamically unfavourable, and binding of nucleic acid probe to the target becomes favoured. This will result in binding of the nucleic acid probe to the target rather than the blocker and thus cleavage of the frangible cross-linker.

Where the target is a nucleic acid, the nucleic acid probe sequence is suitably highly complementary to the target nucleic acid analyte sequence. For example, the sequences of the nucleic acid probe and the target nucleic acid may suitably be perfectly complementary along substantially the entire length of the nucleic acid probe. The nucleic acid probe and the blocker may be complementary along only a portion of the length of the probe (e.g. 90% or less, 80% or less, 70% or less, 60% or less, 50% or less, or 40% or less). Alternatively, or additionally, the nucleic acid probe and blocker may be mismatched at one or more positions along their shared region of partial complementarity (e.g. 1, 2, 3, 4 or 5 mismatched could be present, or, alternatively, 5% or more 10% or more, 20% or more, 30% or more, or 40% or more of bases can be mismatched in the region of partial complementarity).

In one exemplary embodiment, a nucleic acid probe may be from 10 to 100 nucleotides long (preferably from 10 to 80, more preferably from 10 to 50, and yet more preferably from 10 to 30), and may have a region of partial or perfect sequence complementary to its corresponding blocker of from 8 to 20 nucleotides in length.

In a preferred embodiment, the probe sequence is such that it pairs only along part of its length with the blocker strand (e.g. from 20 to 80% of its length, preferably from 40 to 60% of its length), and along a greater proportion of its length with the target; suitably substantially all of its length (e.g. 70% or more, preferably 90% or more, of its length).

The nucleic acid probe and blocker strands are typically oligonucleotides, which can be of any suitable length. Suitably the oligonucleotide comprises 100 or fewer bases, preferably 50, 40, 30, 25, 20 or fewer bases; typically, the oligonucleotide will comprise 10 or more bases.

It should be noted that nucleic acids probes need not be natural nucleic acids (DNA or RNA), but can be a nucleic acid analogue/mimetic or other forms of modified or altered nucleic acids. All that is required is that the nucleic acid probes of the present invention is capable of base pairing with the target nucleic acid. For example, the nucleic acid may be a peptide nucleic acid (PNA), phosphorodiamidate morpholino oligo (PMO) (also known as a Morpholino), locked nucleic acid (LNA), glycol nucleic acid (GNA), Bridged Nucleic Acid (BNA), or threose nucleic acid (TNA). Thus the term ‘nucleic acid probe’ as used herein is not restricted to natural nucleic acids and includes nucleic acid analogue/mimetic and other forms of modified or altered nucleic acids.

It may be advantageous to use a nucleic acid analogue/mimetic that has a reduced charge and/or hydrophilicity compared with equivalent DNA or RNA sequences (as is the case with PNAs and/or Morpholinos). This typically reduces the chance of false positive swelling responses in aqueous environments and thereby improves signal strength and sensitivity to low concentrations of the oligonucleotide marker. Thus, such nucleic acid analogues/mimetics may be present in preferred embodiments of the present invention.

In some embodiments of the present invention the biosensor may comprise nucleic acid probes for the detection of more than one target.

The nucleic acid probe and blocker (and/or blocking target for a nucleic acid aptamer, if present) are preferably linked to a polymerisable moiety. For example, the nucleic acids or blocking target are suitably covalently bound to a monomer moiety which is capable of integration into a swellable polymer. Integration of the polymerisable moiety having nucleic acids/blocking target linked thereto into a suitable polymer results in a cross-linked swellable polymer comprising frangible cross-links.

The polymerisable moiety linked to the nucleic acids may suitably comprise an alkene (olefin) and/or ring-openable group, but it can comprise any other suitable polymerisable group.

The combination of a nucleic acid and a polymerisable moiety can be referred to as a nucleic acid cross-linking monomer, and the combination of a blocking target and a polymerisable moiety can be referred to as a blocking target cross-linking monomer. The generic term cross-linking monomer refers to both unless the context dictates otherwise.

In a preferred embodiment of the present invention the polymerisable moiety linked to the nucleic acid probe and blocker is acrydite. The structure of acrydite-based nucleic acid cross-linking monomer is shown below.

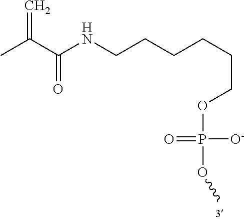

The wavy line represents the oligonucleotide linked to the acrydite moiety. The C═C double bond allows the acrydite to be readily integrated into a polymer backbone during polymerisation, thereby anchoring the oligonucleotide in place within the polymer, and leaving the linked nucleic acid free to cross-link.

The swellable polymer preferably comprises non-frangible cross-links in addition to the frangible nucleic acid cross-links. For example, polyacrylamides can be cross-linked with bisacrylamide, as is well known in the art. If a polymer does not comprise non-frangible cross-links, cleavage of the frangible cross-links can result in the polymer substantially losing its structural integrity, which may be undesirable in many cases. By providing a mixture of frangible and non-frangible cross-links the overall structure of the polymer can be retained, but the swellability of the polymer can be significantly modified depending on the whether the frangible cross-links are cleaved or not.

In use, when a target comes into contact with the swellable biologically sensitive element, it preferentially hybridises (anneals) with the nucleic acid probe and displaces the blocker strand or blocking target. This cleaves the frangible cross-links which results in reduced cross-linking in the polymer. Reduced cross-linking in the polymer allows the polymer to display increased swelling properties.

The person skilled in the art can readily select appropriate levels of frangible and non-frangible cross-linking in a polymer to tune the properties of the polymer. The desired properties and the levels of cross-linking required will depend upon the intended use of the biosensor and the polymer used. Increasing the proportion of non-frangible crosslinking will not only reduce/tune the swelling capability of the system, but may also slow the rate of swelling (by creating a more tortuous/closed path for water penetration. Some non-frangible content is generally preferred to maintain a degree of structural integrity whilst in the swollen state. Generally, in most instances, the total amount of crosslinking would be a maximum of 30%, often 15% or lower, and typical significantly less (e.g. less than 10% or 5%). However, higher levels of cross-linking may be preferred in some situations.

The swellable biologically sensitive element may comprise any suitable swellable polymer, e.g. a hydrophilic polymer. Swelling of the polymer is typically the result of the interaction of the polymer with water (or other suitable, e.g. polar, solvent) present in the sample. The swellable biologically sensitive element is adapted to swell to a suitable (i.e. detectable) extent when exposed to a suitable sample which comprises the target analyte, e.g. nucleic acid, (i.e. upon cleavage of the frangible cross-linkers), but does not swell or swells significantly less when the target analyte (e.g. nucleic acid) is not present.

In many cases the swellable biologically sensitive element will swell to some extent in the presence of water (or other suitable solvent), but in the absence of the target analyte. However, any such swelling is constrained by the nucleic acid probe present in the swellable biologically sensitive element (e.g. in the form of frangible cross-linkers). When the frangible cross-linkers are cleaved, the constraints on the swelling of the polymer are reduced, and thus swelling capability of the swellable biologically sensitive element is increased. Thus, the swellable biologically sensitive element is suitably differentially swellable depending on the presence or absence of the target analyte.

In some cases, the swellable biologically sensitive element may be ‘pre-swollen’ prior to use by exposing the swellable biologically sensitive element to a suitable aqueous liquid which has similar or identical properties to the sample to be analysed; in this case a significant change in the properties of the polymer during use would only occur in the presence of the target analyte. Alternatively, data obtained from the biosensor can be processed to disregard any background swelling which occurs absent the target analyte.

As mentioned above, swelling occurs as a result of absorption of water (or other suitable solvent) present in a sample by the swellable biologically sensitive element. Accordingly, the sample to be analysed will typically be liquid, and will usually be aqueous. The sample can suitably be, or be derived from, a biological sample from an animal, e.g. blood, urine, saliva, mucous, faeces, lymphatic fluid, CSF, synovial fluid, or the like. Alternatively, the sample could be any other source which might comprise an analyte of interest, e.g. cell culture medium, environmental samples, etc. The sample can comprise a diluent, e.g. water, saline or a buffer.

Hydrophilic polymers are a particularly suitable material for use in the swellable biologically sensitive element. As is well known in the art, the water absorption (and thus swelling) properties of hydrophilic polymers, such as hydrogels, are significantly determined by the amount of cross-linking present (sometimes known as cross-linking density). Accordingly, in preferred embodiments of the present invention, the swellable biologically sensitive element suitably comprises a hydrophilic, water-swellable polymer.

The hydrophilic polymer is preferably cross-linked (i.e. contains conventional cross-links in addition to frangible nucleic acid cross-links, as discussed above). Hydrophilic polymers, especially when cross-linked, are often referred to as hydrogels. Accordingly, the biosensor of the present invention preferably comprises a swellable biologically sensitive element comprising a hydrogel.

There are many hydrophilic polymers known in the art, and the person skilled in the art could readily select an appropriate hydrophilic polymer for use in the present invention.

To give some particularly suitable, but non-limiting, examples, the hydrophilic polymer may comprise one or more of polyacrylamides, poly(N-isopropylacrylamide)s; poly(vinyl alcohol)s; poly(ethylene glycol)s; poly(N,N′-dialkyl acrylamide)s; poly(2-hydroxypropyl (meth)acrylate)s; poly(2-methoxyethyl acrylate)s; and poly(di(ethylene glycol)methyl ether methacrylate)s. Integration of frangible nucleic acid cross-linkers into such polymers can be readily achieved by using appropriate polymerisable moieties linked to the nucleic acids.

The polymer preferably comprises a polyacrylamide hydrogel. The polymer normally comprises a monomer (acrylamide) and a cross-linker (suitably N,N′-methylene-bisacrylamide). The polymer may suitably comprise any suitable proportion of acrylamide, e.g. from 5 to 50%, preferably from 7 to 30%, typically from 10 to 20%, and usually 10-15% (e.g. approximately 15%) w/w acrylamide. The concentration of the cross-linker in the polymer may be varied to tune the physical characteristics of the resultant hydrogel. The concentration of the cross-linker in the polymer can be present at any suitable level, e.g. it may suitably be present at from 0.05 to 10 mol %, preferably from 0.1 to 5 mol %, yet more preferably from 0.2 to 2.0 mol % with respect to the monomer.

The polymer may suitably comprise a photoinitiator. The photoinitiator may suitably be 1-hydroxycyclohexylphenylketone. However, many other photoinitiators are well-known and other examples include: AIBN and other azo compounds; benzoyl peroxide and other peroxides; 2,2-dimethoxy-2-phenylacetophenone; and camphorquinone, and further photoinitiators are available from Sigma Aldrich (see: https://www.sigmaaldrich.com/content/dam/sigma-aldrich/docs/Aldrich/General_Information/photoinitiators.pdf). The concentration of the photoinitiator may be present at any suitable concentration, e.g. from 0.05 to 0.5 mol %, preferably from 0.1 to 0.2 mol %, typically 0.125 mol % with respect to the monomer. The photoinitiator typically serves to enable polymerisation to be initiated by UV radiation.

Other types of polymerisation initiators are well known in the art, and can be present as an alternative (or addition) to photoinitiators. For example, initiators for radical polymerisation can include peroxides, persulfates, metal-alkyl, azo or other related compounds. Such initiators thus include thermo-initiators and redox initiators, in particular. Examples of suitable initiators are well known in the art. Such initiators may be present at any suitable concentration, e.g. from 0.05 to 0.5 mol %, preferably from 0.1 to 0.2 mol %, typically 0.125 mol % with respect to the monomer. For example, persulfates, e.g. ammonium persulfate (APS), in the presence of diamines, e.g. tetramethylethylenediamine (TEMED) can be used as a redox initiator. For example, 0.125 mol % with respect to monomer has been used successfully. Polymerisation can also be initiated by other strategies including coordination-insertion, anionic, cationic, controlled radical, ring-opening and other polymerisation techniques depending upon the monomer and end-functionality chosen.

The polymer may suitably comprise sodium chloride or another inorganic metal salt. This is useful for stabilising DNA or other charged nucleic acid probes/cross-linkers incorporated into the polymer, but would typically not be required when Morpholino or PNA frangible probes/cross-linkers are used. The concentration of the sodium chloride may suitably be 0 to 500 mM, with approximately 150 mM being typical.

The polymer may suitably comprise a solvent, or a solvent may be used during polymerisation of the polymer. In particular, the solvent can be used as an aid to uniformly distribute conductive particles in the polymer. The solvent may comprise dimethyl sulfoxide (DMSO) and a phosphate buffer. The solvent may comprise an equal volume of dimethyl sulfoxide (DMSO) and phosphate buffer. The phosphate buffer can have any suitable concentration, and in some cases has a concentration of about 1 mM (millimolar).

The polymer may suitably be a copolymer. Copolymerisation can be useful as it allows for tuning of the properties of the final polymer. For example, a suitable copolymer can comprise acrylamide and one or more additional monomers (in addition to the monomers to which the nucleic acids are linked). The one or more additional monomers may be hydrophilic or may be non-hydrophilic, provided that the copolymer is hydrophilic overall. The one or more additional monomers may be one or more of acrylates, methacrylates, vinyl acetates, styrenes, acrylonitriles and olefin-containing monomers.

Preferably the polymer comprises proportionally at least 60 mol % (more preferably at least 80% and yet more preferably at least 90 mol %) acrylamide monomer and 40% or less (more preferably 20% or less and yet more preferably 10% or less) of additional monomer(s).

In one specific example, the polymer can comprise acrylamide and N-Isopropylacrylamide monomers. For example, polymers having 50:50, 80:20 and 20:80 ratios of acrylamide to N-Isopropylacrylamide according to the present invention have been successfully prepared.

In preferred embodiments of the present invention the swellable biologically sensitive element comprises an oligonucleotide cross-linked polymer composite (OCPC).

The physicoelectrical transducer of the present invention is able to convert a physical change in the swellable biologically sensitive element into an electrical signal or electrical property which can be detected.

Preferably, the physicoelectrical transducer of swelling comprises a piezoresistive material or electrically percolating composite material (see below for definition of a “percolating composite”), capable of exhibiting changes in its electrical impedance due to externally-induced changes in its volume (e.g. mechanical deformation or chemical swelling). In the case where electrical resistance change is the property to be detected, such transducers can be referred to as “chemi-resistive” (or perhaps in this case “bio-resistive”). Alternatively, other forms of physicoelectrical transducers can be used, e.g. those comprising piezoelectric materials.

The preferred type of physicoelectrical transducer to which this patent refers are commonly referred to as “percolating composites”. Herein, we define this term as a material with the following properties: comprising of a blend of two or more discrete elements with differing electrical conductivities; exhibiting a decrease in electrical conductivity as the effective concentration of the more conductive element is decreased; and exhibiting a decrease in electrical conductivity upon volumetric swelling.

Typically, percolating composites comprise electrically conductive particles (e.g. carbon or metal powders, as discussed further below), dispersed within an insulating polymer matrix (in this case a swellable polymer matrix). At low filler loadings of the conductive particles the composite is electrically insulating, with a conductivity (or resistivity) close to that of the polymer. As the concentration of conductive particles is increased, more particles come into physical contact with each other, and the conductivity rises rapidly over a narrow concentration range (the so-called “percolation threshold”). It then asymptotically approaches the conductivity of the metal at high filler particle concentrations. An approximately sigmoidal pattern of conductivity (resistivity) with conductive particle loading is therefore observed for such materials. A similar pattern of behaviour is also observed when the polymer is mechanically compressed or chemically swollen, whereby the volume of the polymer is changed and thus alters the effective concentration of conductive particles.

These types of materials are commonly referred to as “percolating composites”. This is due to the fact that “Percolation Theory” was first proposed to describe such patterns of behaviour. However, this model is too simplistic for many composites and fails at low concentrations, since it predicts no conduction (infinite resistance) in this region. The percolation model is also ineffective when used to describe the behaviour of blends of conductive and non-conductive polymers, and indeed any two materials with a finite difference in their conductivities. “Effective Medium Theories” have since been successfully devised to provide a more accurate description of the electrical behaviour across the full range of conductive particle loadings. They can also be used to model how the particle shape and size affect the percolation threshold. However, some materials still have properties not well predicted by either Percolation Theory or Effective Medium Theory, and need further modifications to the theory. These notably include “Quantum Tunnelling Composite” (or “QTC”), which displays no percolation threshold with filler concentration, yet extreme sensitivity to volumetric changes through compression or swelling, and does not rely upon intimate contact between adjacent particles to enable conduction through the composite (relying instead upon quantum tunnelling between the particles).

It is important to note that throughout this document (and indeed throughout much of the literature), the term “percolating composite” is used to describe all above examples of these types of materials following the previously described definition, despite the large number of examples that do not strictly conform to the model of Percolation Theory.

The percolating composite of the physicoelectrical transducer is thus adapted for the transduction and/or conversion of swelling of the swellable biologically sensitive element into a measurable change in electrical impedance. In the present invention the electrical resistance of the or percolating composite changes when the swellable biologically sensitive element swells, which occurs in the presence of the appropriate target analyte (e.g. oligonucleotide) and water (or other suitable solvent). As discussed earlier, while some swelling may occur in the absence of the target analyte in some cases, this is less than the swelling which occurs in the presence of the analyte, which permits the presence and/or amount of the analyte to be detected.

The percolating composite suitably comprises an electrically conductive material (such as a metal or carbon) or a semiconductor, which is preferably in particulate form.

The percolating composite preferably comprises conductive particles distributed in the swellable biologically sensitive element. For example, the hydrophilic polymer of a swellable biologically sensitive element may define a matrix in which conductive particles are distributed. The conductive particles can be referred to as a conductive filler or conductive filler particles.

It will be appreciated that the conductive particles distributed in the swellable biologically sensitive element will be subjected to movement as the swellable biologically sensitive element swells and increases in volume. Typically, as the swellable biologically sensitive element swells the packing density of the conductive particles will decrease, i.e. the spacing between each particle will increase. This results in an increase in electrical resistance in the material. This change in resistance can be readily detected, e.g. by the use of suitable electrodes connected to the swellable biologically sensitive element.

Highly conductive particulate materials are preferable to maximise the electrical conductivity change upon swelling. High conductivity, e.g. as demonstrated by metal or carbon particles, is preferred as it maximises the resistance change upon swelling of the swellable biologically sensitive element. In general, any highly conductive particles of an appropriate size can be used.

The percolating composite preferably comprises carbon nanopowder and/or carbon nanotubes as the conductive particles.

Carbon nanopowder in many forms can be used. The present inventors have found that carbon nanopowder having an average particle size of less than or equal to 50 nm is eminently suitable, but this is not restrictive of the present invention. Likewise carbon nanotubes in many forms can be used. The present inventors have found that carbon nanotubes which are multi-walled and which have an outer diameter of approximately 10 nm and are approximately 6 μm long are eminently suitable, but this is not restrictive of the present invention. The skilled person could readily select alternative carbon nanopowder or nanotube materials.

The percolating composite may alternatively or additionally comprise one or more of carbon black, nickel, gold, silver, zinc and copper particles. Other electrically conductive metallic particles can of course be used, as could any other suitable conductive non-metallic particles, including conductive polymer particles/beads.

It will be apparent to the skilled person that combinations of two or more of the above conductive particulate materials can be used.

It is preferred that the conductive particles are sub-micrometre in size, e.g. less than 1000 nm, preferably less than 500 nm, and more preferably less than 100 nm. Such particles are preferred for their greater compatibility with large-scale printing processes, such as ink-jet printing. Smaller filler particles also permit the further miniaturisation of swellable biologically sensitive element volumes (which can allow for faster response), whilst maintaining uniform particle distribution and good associated electrical repeatability.

As an alternative to conductive filler particles, the skilled person could also use intrinsically conductive polymers, which could be blended with the abovementioned (insulating) polymer materials. Examples of conductive polymer fillers include, but are not restricted to: poly(thiophene) (PT), poly(3,4-ethylenedioxythiophene) (PEDOT), poly(p-phenylene sulphide) (PPS), poly(aniline)s (PANI), poly(pyrrole)s (PPY), poly(acetylene)s (PAC), poly(p-phenylene vinylene) (PPV), and mixtures and blends thereof.

Where alternating current (AC) electrical impedance measurements are utilised for electrical measurement of response (see below for more details), the filler particles can suitably be used to tune the dielectric properties of the composite (and hence both its resistance and complex impedance) to optimise the measurable response to swelling. In this instance, it may be preferable to lower or raise the impedance of the composite to a region with greatest sensitivity. In these instances, other conductivities of filler particles may be preferable, and may include semiconductor powders, or even insulating particles.

The person skilled in the art can readily select appropriate conductive materials from those set out above. Furthermore, the skilled person can provide the conductive materials appropriate quantities to achieve desired percolation properties in the biosensor, i.e. appropriate changes in impedance when the polymer swells in the presence of the target analyte. It is neither possible nor necessary to provide binding general rules about the relative proportions of swellable polymer to conductive particles as it will vary from case to case. However, it would be a simple matter for the skilled person to optimise any given system.

The proportion of conductive particles is preferably such that before swelling, the composite is above the percolation threshold and is therefore in a maximally conductive state. Then, after swelling has occurred, the composite will be become non-percolating and in a minimally conductive state. By loading the conductive powder at a value close to this ‘percolation threshold’, sensitivity of the sensor is maximised. (The percolation curve has a sigmoidal shape, and thus resistance changes very sharply at the percolation threshold).

However, in some cases it may be preferred to choose to have an initial state slightly further away from the percolation threshold, so that initial (false positive) swelling from the solvent alone (i.e. absent the target analyte) has minimal effect upon resistance. The additional triggered swelling in the presence of the target analyte, and resultant cross-linker cleavage, then pushes the composite beyond the percolation threshold for detection.

In either case, the sensor should be at a filler volume fraction somewhat above percolation immediately before exposure to the target analyte. Exposure then swells the composite, changing the volume fraction to a value below the percolation threshold.

Percolation threshold concentrations are understood well in the literature, and are determined by particle size, shape and aspect ratio.

The percolation threshold thus provides a convenient mechanism to intrinsically amplify changes in resistance which result from swelling (i.e. an increase in volume). The biosensor of the present invention is suitably adapted such that swelling of the polymer upon cleavage of the nucleic acid cross-linkers results in a change in volumetric fraction of the conductive particulate material within or across (or across at least part of) the percolation threshold. The percolation threshold for any combination of conductive particulate material and polymer can be readily determined.

The biosensor of the present invention typically comprises a substrate comprising suitable electrodes to allow detection and/or measurement of the electrical signal or change in electrical properties of the biosensor as a result of swelling of the swellable biologically sensitive element.

Any suitable electrode system can be used. The present inventors have found interdigitated electrodes (IDEs) to be particularly suitable. The electrodes can be provided on a silicon substrate, e.g. a silicon wafer. Fabrication of such electrodes can be carried out using standard photolithography procedures, i.e. patterning metallic electrodes onto silicon substrates.

In one exemplary embodiment of the present invention the electrodes comprise platinum IDEs fabricated upon a silicon dioxide (SiO₂) substrate. An adhesion layer, e.g. formed of titanium, can also be used to assist with adhesion of the platinum to the SiO₂. In another exemplary embodiment gold electrodes can be patterned onto glass substrates. It will be apparent that many other suitable electrode systems can be used, and, in general, any insulating material, with conductive patterned electrodes would be suitable. For many applications it may be preferred that the electrode is disposable and low cost, consisting perhaps of plastic or paper substrates, patterned with printed metal, carbon or conductive ink electrodes; such electrodes are already known in the art.

Suitably the active area of the electrodes is completely covered with the swellable biologically sensitive element (e.g. OCPC).

In some embodiments the substrate is at least partially surface modified in the area where the swellable biologically sensitive element is to be deposited with an agent to enhance adhesion of the swellable biologically sensitive element to the substrate and/or electrode. The substrate can suitably silanised, e.g. with 3-(trimethoxysilyl)propyl methacrylate. Silanisation is particularly useful when glass or silicon substrates are used.

Suitably the biosensors of the present invention are single use items.

In accordance with a second aspect of the present invention there is provided an array of biosensors according to the present invention on a substrate. This allows for the performance of a plurality of sensing operations to be performed in parallel on a single array.

The biosensors in the array can be configured to detect a plurality of target analytes, and/or can be configured to carry out more than one parallel detection for the same target (i.e. multiple replicates). Detection of several different targets is clearly desirably to efficiently screen for a plurality of targets, and multiple replicates may be desirable to increase statistical significance/confidence or eliminate noise.

Multiple replicates can also be used to obtain quantitative information about target concentration. This is achieved by varying cross-linking density between several different sensors, but using the same probe in each. The differential response magnitude and differential temporal response of this group of sensors will then enable quantitative information to be obtained about the relative abundance of the target species.

Multiple sensors, each with different degrees of target-probe overlap/match for a single specific target, could also be employed for improving selectivity. This would help to identify and ignore instances of false positive responses from sensing events where a more energetically favourable state results from the probe binding to the target, but yet the probe and target are still not 100% matched.

Suitably the array comprises 2, 3, 4, 5, 6, 7, 8, 9, 10 or more biosensors. However, there is effectively no limit to the number of biosensors which can be provided on the array, and thus the array may suitably comprise 20 or more, 50 or more, 100 or more, or 500 or more biosensors. Potentially the array could be configured to detect a small or large panel of miRNAs of clinical or research significance. For example, the array could comprise several miRNAs, which are indicative of a specific disease (i.e. for diagnosis) or disease state (i.e. for stratification or for treatment monitoring). Alternatively, the array could comprise a much wider range of miRNAs, for multiplexed screening of multiple diseases.

The substrate suitably comprises suitable electrodes to interact with each of the biosensors.

In a third aspect the present invention provides a biosensor device comprising a biosensor or array according to the first or second aspects of the invention connected to a measurement apparatus adapted to detect and/or quantify an electrical indicator from the biosensor.

The measurement apparatus can be adapted to measure a change in impedance (e.g. using an ohmmeter) or electrical signal, typically a voltage (e.g. using a voltmeter), from the biosensor. Suitable sensitive metering apparatuses are well known in the art.

The biosensor device can suitably comprise a sample receiving vessel to receive a sample to be analysed. The biosensor or array is adapted to be exposed to a sample in the vessel.

In accordance with a fourth aspect of the present invention there is provided a method for detecting the presence or amount of a target in a sample, the method comprising:
a) providing a biosensor according to the first aspect of the present invention or an array according to the second aspect of the present invention;
b) exposing the biosensor to the sample;
c) observing for an electrical signal or change in electrical property in the biosensor as a result of exposure to the sample;
d) optionally determining the magnitude of said change; and/or
e) optionally determining the speed of said change; and
f) determining therefrom the presence and/or amount of said target in said sample.

The target can be a nucleic acid or any other suitable target when an aptamer probe is used.

Preferably the method is for the detection of a target (e.g. an oligonucleotide such as miRNA) in a biological sample. However, the method can be applied to any suitable sample in which the target might be present. Preferably the sample is aqueous.

The method may involve the step of obtaining the sample from a subject, or it could be performed on a pre-obtained sample.

Preferably where the sample is obtained from a subject, the subject is a mammal; more preferably the subject is a human.

Suitably the method is for the detection of more than one target analyte in the sample. For example, a plurality of nucleic acids (e.g. miRNAs) can be detected in a single method, a plurality of non-nucleic acids can be detected, or a combination of nucleic acids and non-nucleic acids can be detected. This typically requires the use of a plurality of biosensors adapted to detect different targets.

The method is suitably a diagnostic method, and it can be used to assist in diagnosing a medical condition and/or selecting an appropriate therapy for the subject.

Alternatively, the method be used for monitoring recovery or response to a therapeutic agent.

In a fifth aspect of the present invention, there is provided a method of producing a biosensor, the method comprising:
a) preparing a swellable biologically sensitive element comprising a nucleic acid probe; and
b) associating a physicoelectrical transducer with the swellable biologically sensitive element.

In preferred embodiments the swellable biologically sensitive element comprises a swellable polymer, suitably a hydrophilic polymer, for example a hydrogel. Particularly preferred polymers and copolymers, and the associated monomers for their formation, are set out above. Accordingly, the method suitably comprises providing suitable monomers to form the swellable polymer (e.g. acrylamide monomers) and polymerising said monomers to form a polymeric swellable biologically sensitive element.

The method preferably comprises providing nucleic acid cross-linking monomers and integrating said a nucleic acid cross-linking monomers into the polymer. Suitably the nucleic acid cross-linking monomers comprise a nucleic acid linked to acrydite. Preferably the method comprises annealing corresponding nucleic acid cross-linking monomers prior to incorporation into the swellable polymer. Suitable nucleic acid cross-linking monomers are discussed above, and others will be apparent to the skilled person.

Suitably the method comprises providing non-frangible cross-linkers and integrating said non-frangible cross-linkers into the polymer. Suitable non-frangible cross-linkers are well known in the art, and some are discussed above while others will be apparent to the skilled person.

Accordingly, the method suitably comprises providing suitable monomers to form a swellable polymer matrix (e.g. acrylamide monomers), providing nucleic acid cross-linking monomers, optionally providing non-frangible cross-linkers, and causing the monomers and optionally non-frangible cross-linkers to polymerise, thereby forming a swellable polymer comprising frangible nucleic acid cross-links.

Polymerisation of the monomers can suitably be achieved by providing a polymerisation initiator and initiating polymerisation by applying suitable conditions, e.g. by photoinitiation, thermal initiation, or suchlike. Suitable initiators are well known in the art, and examples are discussed above.

Preferably the method comprises providing electrically conductive particles and distributing said electrically conductive particles in the swellable biologically sensitive element, preferably to form a percolating composite. For example, the electrically conductive particles are suitably mixed with monomers prior to polymerisation of the monomers to form a swellable polymer. Suitable conductive particles are discussed above, as are the preferred properties of percolating composite materials.

Accordingly, the method suitably comprises:
providing suitable monomers to form a swellable polymer,
providing nucleic acid cross-linking monomers,
providing electrically conductive particles,
optionally providing non-frangible cross-linkers,
mixing the aforementioned together, and
causing the monomers, and optionally non-frangible cross-linkers, to polymerise, thereby forming a swellable polymer comprising frangible nucleic acid cross-links having an associated a physicoelectrical transducer. Preferably the method produces a percolating composite.

Suitably the method comprises the steps of providing a substrate comprising electrodes and covering the electrodes on said substrate with the swellable biologically sensitive element and associated physicoelectrical transducer. Suitably the swellable biologically sensitive element is a polymer, and the method comprises polymerising suitable monomers, and optionally crosslinkers, to form the polymer in situ on the substrate.

The optional features of the first aspect of the present invention can be incorporated into the second, third, fourth and fifth aspects of the present invention and vice versa.

Embodiments of the present invention will now be described by way of example only and with reference to the accompanying drawings, in which:

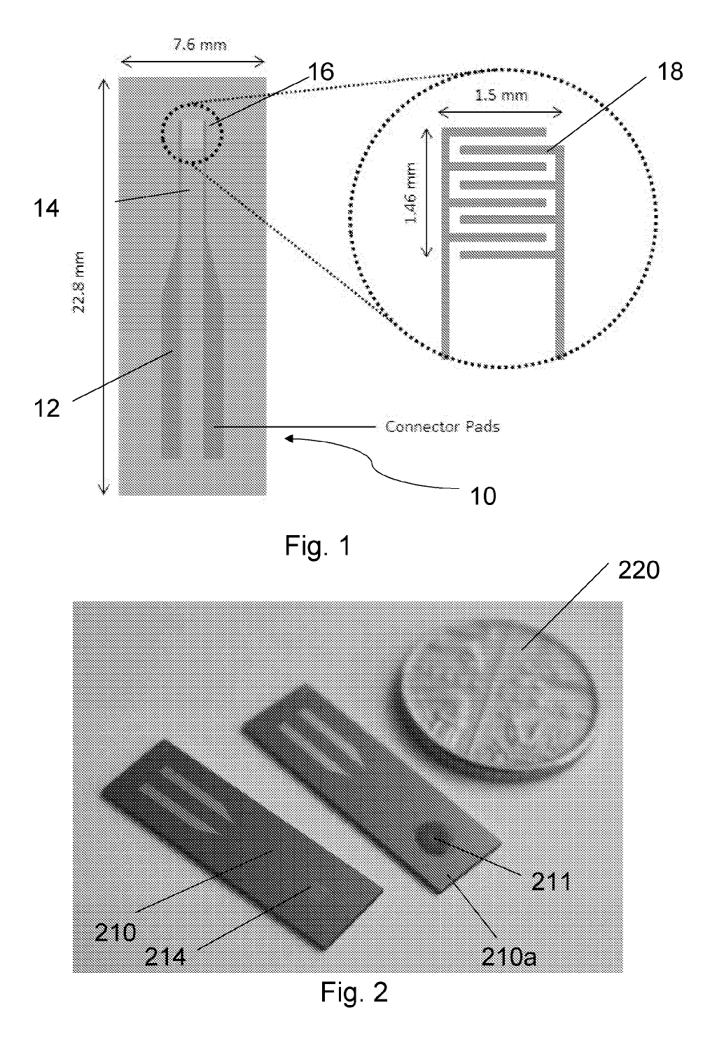

FIG. 1 is a schematic view of a biosensor according to an aspect of the present invention;

FIG. 2 is an image of a fabricated microelectrode chip;

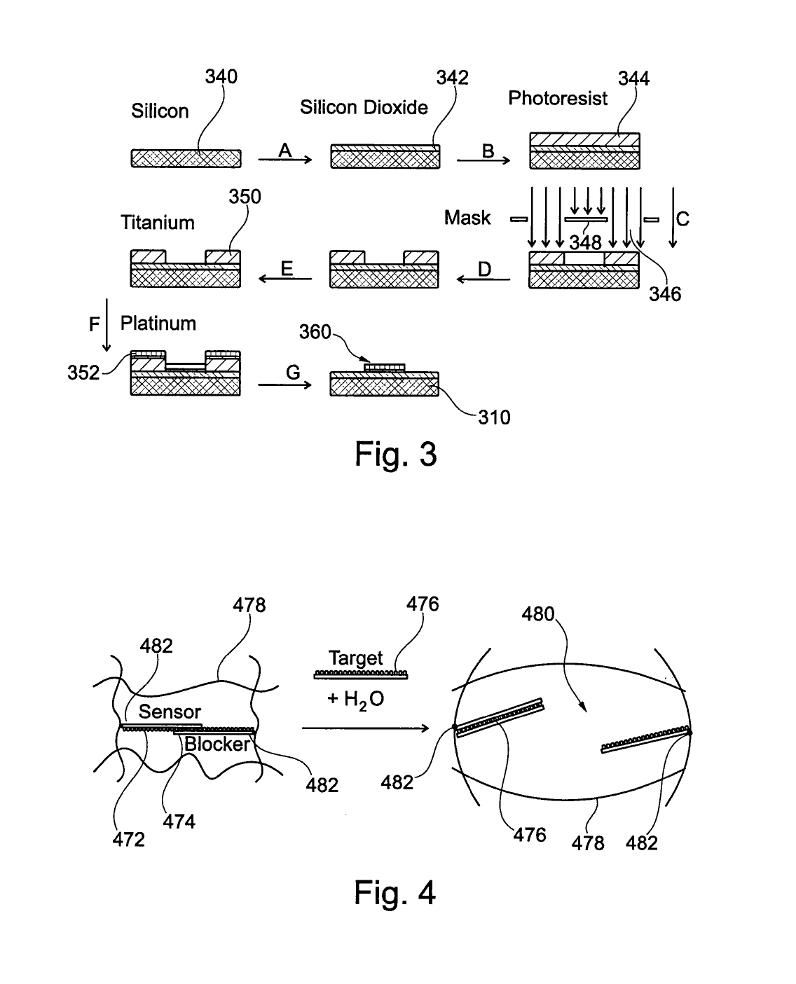

FIG. 3 is a schematic view of the fabrication steps of the electrodes;

FIG. 4 is a schematic view of the swelling response of an oligonucleotide functionalised hydrogel;

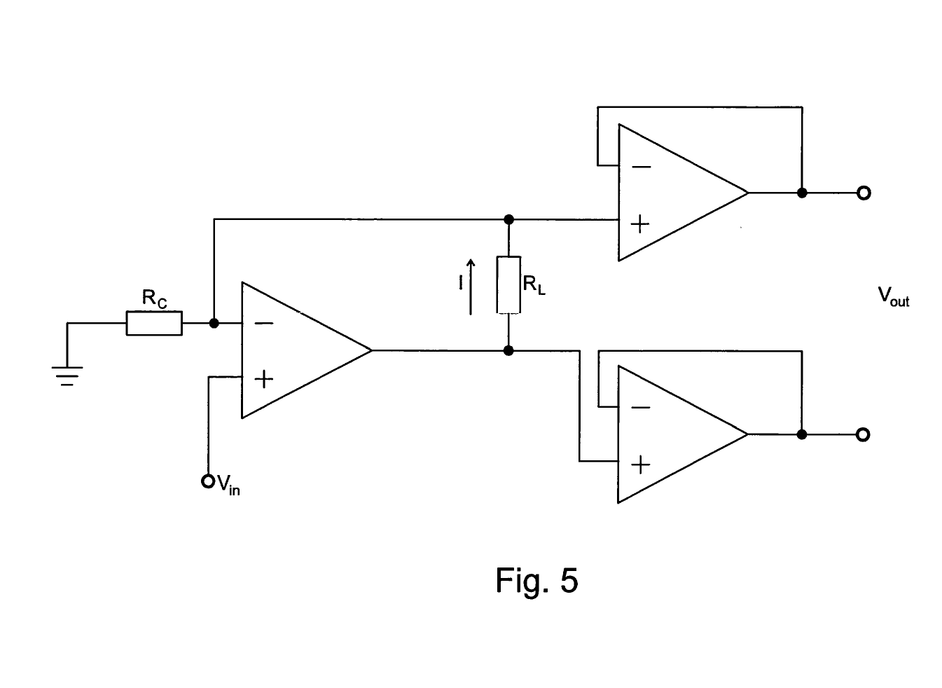

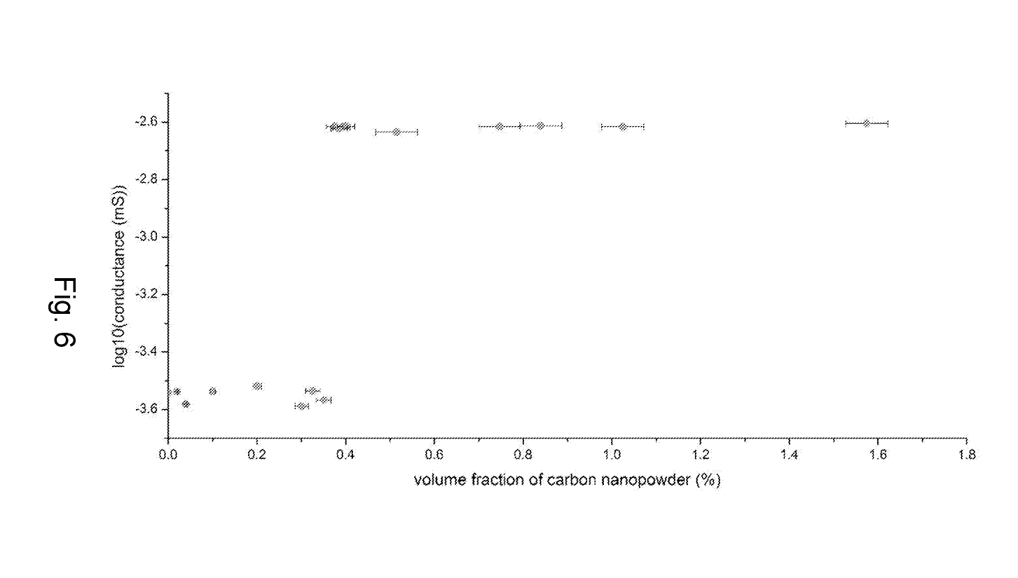

FIG. 5 is a schematic view of the circuit diagram;

FIG. 6 is a graph of the how the conductance (i.e. 1/resistance) varies as the volume fraction of conductive filler is varied (i.e. it shows the percolation threshold for a particular composite);

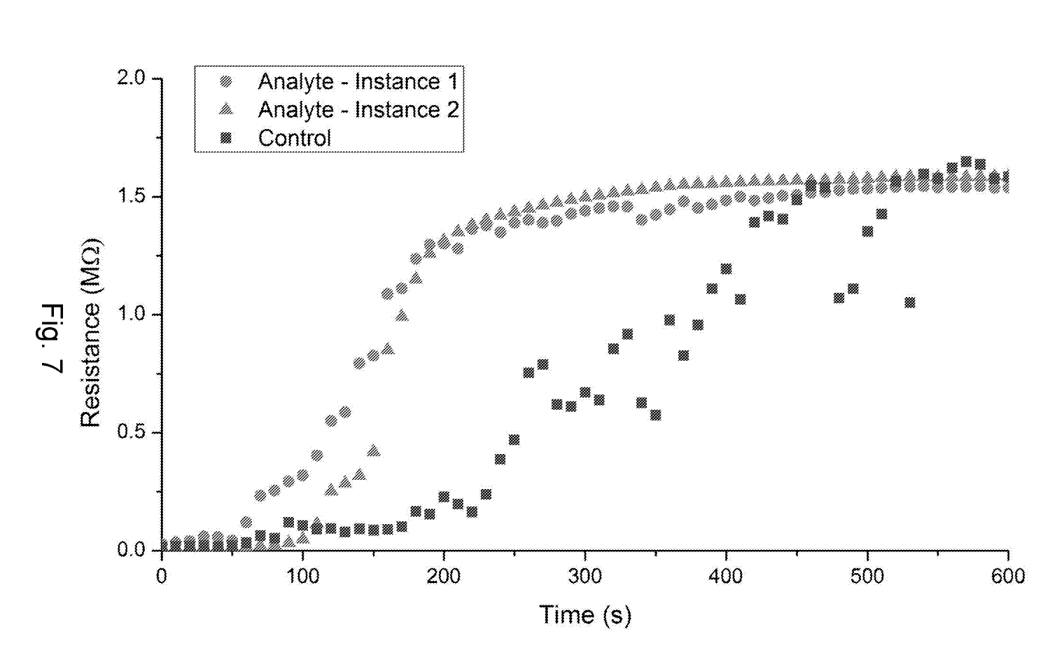

FIG. 7 is a graph of the resistance of OCPCs;

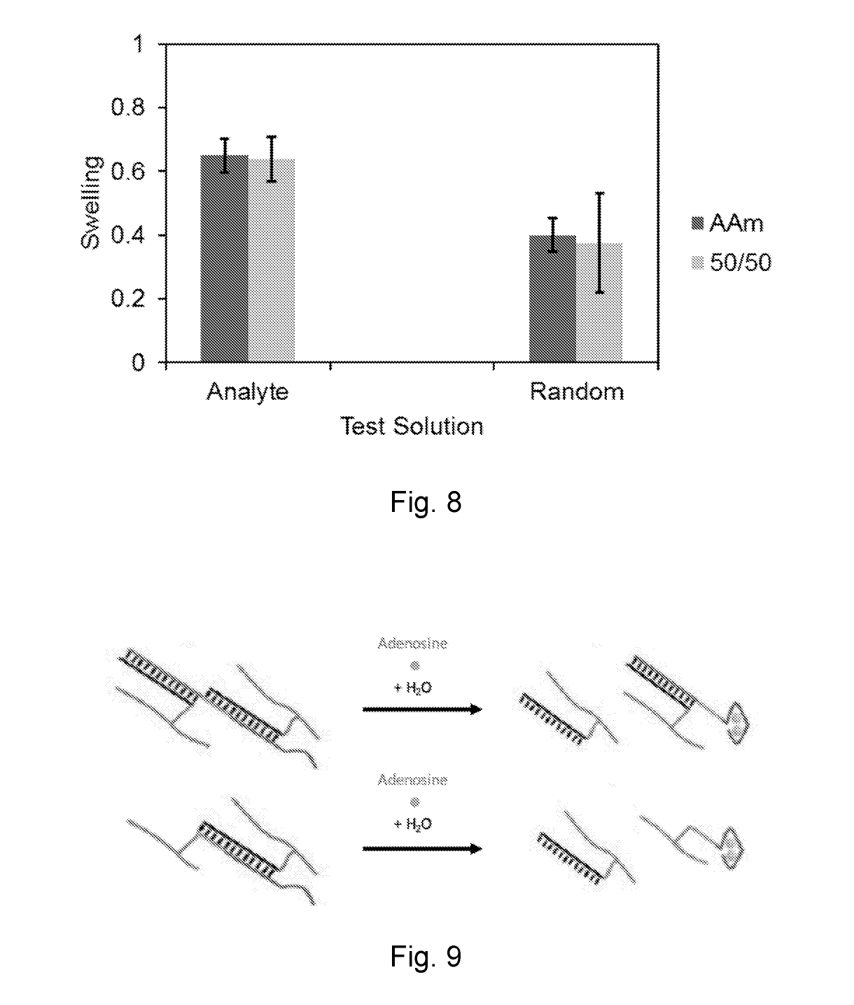

FIG. 8 shows a comparison of swelling in OCPCs containing acrylamide (AAm) monomers and a 50/50 copolymer with N-isopropylacrylamide (NIPAAm) and AAm;

FIG. 9 is a schematic of an OCPC containing aptamer frangible crosslinks. The DNA aptamers bind to adenosine. Two approaches were used, as represented in the upper and lower panels of the figure;

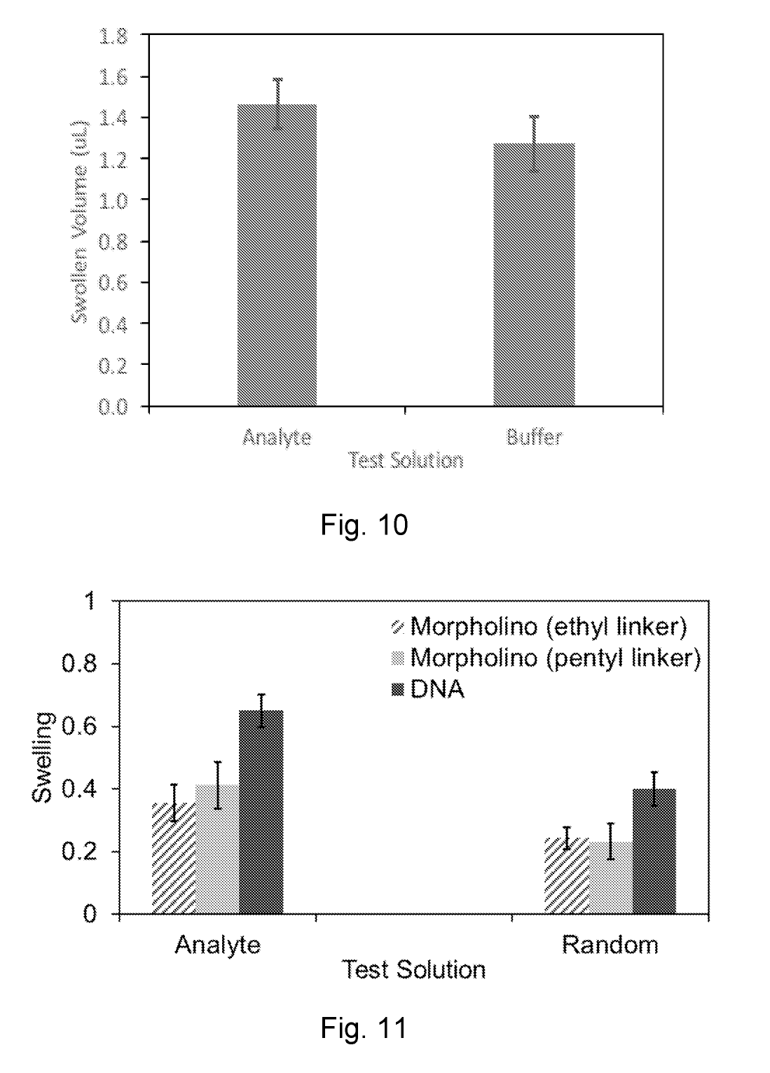

FIG. 10 is a graph showing swelling of OCPCs containing aptamers. The graph shows and aptamer OCPC according to Approach 2, i.e. containing SEQ ID NO:12 and SEQ ID NO:11. The test analyte is 2 mM adenosine in 1 mM PBS and 300 mM NaCl. Buffer is 1 mM PBS and 300 mM NaCl;

FIG. 11 is a graph showing the swelling results OCPCs containing morpholino-based frangible crosslinkers linked to the polymer backbone in different ways (ethyl- and pentyl-linked) compared to DNA-based frangible crosslinkers;

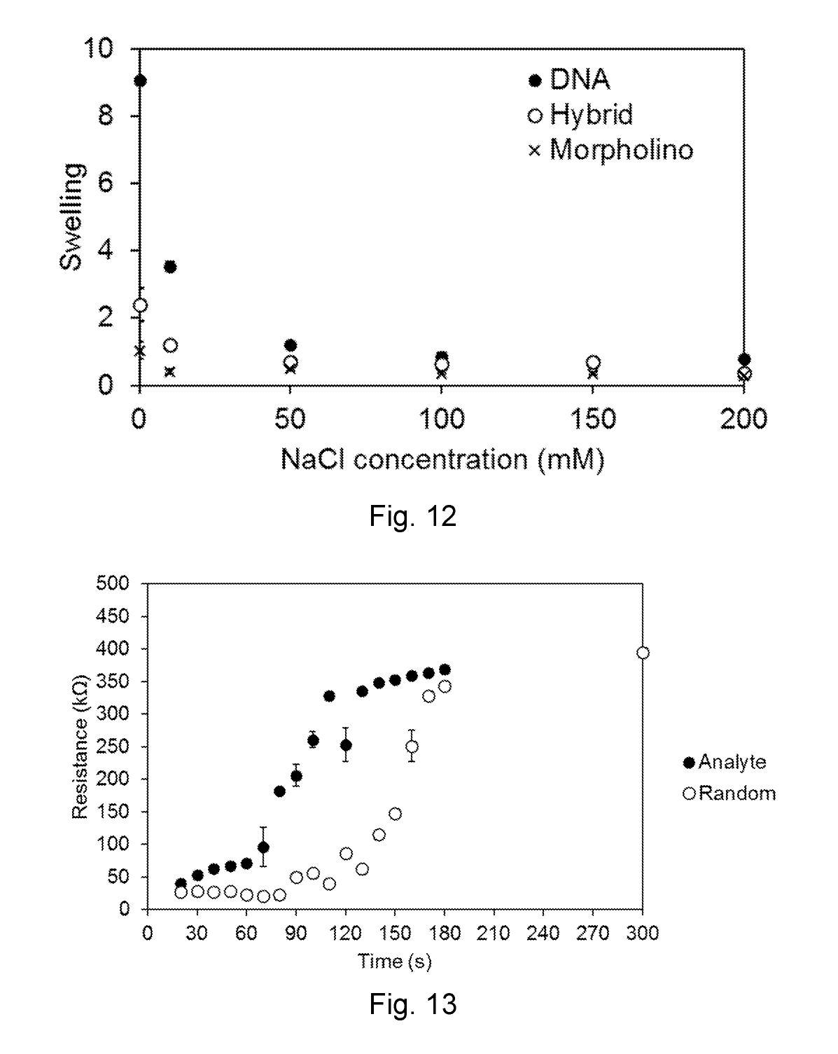

FIG. 12 is a graph showing the salt sensitivity of OCPCs containing morpholino versus DNA sensor and blocker strands, and hybrids containing a morpholino sensor strand and a DNA blocker strand; and

FIG. 13 shows a graph of the resistance of OCPCs produced without the use of DMSO.

The structure and manufacture of an exemplary biosensor is described below.

### Electrodes

The biosensor comprises an interdigitated electrode (IDE). Fabrication of the electrode is carried out using standard photolithography procedures, patterning metallic electrodes onto silicon substrates, as described below. FIG. 1 is a schematic representation of the electrode 10. The electrode 10 may be referred to as an electrode chip. Area 12 is platinum (Pt), area 14 is silicon dioxide (SiO₂). The circled area 16 shows the interdigitated electrodes 18. There are 30 fingers in total (not all are shown for clarity).

FIG. 2 shows the fabricated microelectrode chips 210 on silicon substrates 214. Also shown is a chip 210a deposited with an Oligonucleotide Cross-linked Polymer Composite (OCPC) 211. A 5 pence coin 220 is shown for size reference.

The fabrication steps of the electrodes 310 are shown in FIG. 3. The relevant fabrication techniques are well known in the art. The electrodes 310 used were platinum (Pt) IDEs fabricated upon silicon dioxide (SiO₂). The chips were designed such that the IDEs would be completely covered by the droplets of the OCPC and as such the IDE array spans 1.5 by 1.46 mm. There are 30 fingers in total in the IDE, separated by 40 μm with each one being 10 μm in width (as shown in FIG. 1).

The electrodes 310 were fabricated upon 100 mm silicon wafers 340. These were put through a wet oxidation furnace at 1100° C. for 40 minutes in order to produce a SiO₂ layer 342 of approximately 500 nm. After this the wafers 340 were put in a barrel-ash for one hour in order to remove any traces of moisture from the surface (30 minutes is another suitable period of time that has been successfully used).

After an insulating SiO₂ layer 342 is grown upon the silicon wafer 340, a layer of photoresist 344 is deposited. The wafer is then exposed to UV light 346 through a photolithography mask 348. The photoresist 344 is then developed and titanium 350 is evaporated onto the wafer. Platinum 352 is then evaporated onto the wafer and a lift-off process is performed, leaving only the desired pattern 360.

Prior to the deposition of the photoresist the wafers were primed with hexadimethylsiloxane (HMDS) for approximately 10 minutes to improve the adhesion of the photoresist to the surface of the SiO₂. The photoresist used was AZNL of 2070-3.5 and it was deposited at a thickness of approximately 3.5 μm using a Polos Spinner with a spin profile of 700 revolutions per minutes (rpm) for 5 seconds followed by 3000 rpm for 45 seconds, with an acceleration of 1000 rpm/s. After deposition of the photoresist the wafers were soft-baked on a hotplate at 100° C. for 5 minutes in order to remove any remaining solvent.

The photoresist coated wafers were then exposed to ultraviolet (UV) radiation using a Karl Suss mask aligner and the aforementioned mask for 30 seconds under hard contact (FIG. 3-C). After exposure the wafers were transferred to a hotplate for a reverse-bake at 115° C. for 1 minute. The wafers were then placed in AZ726 developer solution for 1 minute, before being transferred to a fresh batch of developer for a further minute (FIG. 3-D).

The metal was then deposited on the wafers using an e-beam evaporator. Titanium (Ti) was deposited at a thickness of 10 nm (FIG. 3-E) to act as an adhesion layer and Pt was deposited on top at a thickness of 100 nm (FIG. 3-F) (50 nm is another thickness of deposition layer that has been successfully used). After this the lift-off process was performed by immersing the wafers in NMR 1150 at 50° C. for 1 hour, before being transferred to a fresh batch of NMR 1150 (again at 50° C.) for another hour (FIG. 3-G). The wafer was then immersed in isopropyl alcohol (IPA) for 15-20 minutes before rinsing with deionised (DI) water. After the lift-off step, the wafers were then diced into the individual chips.

In order to improve adherence of the OCPC to the electrode surface as it swells, the electrodes were silanised with 3-(trimethoxysilyl)propyl methacrylate in order to chemically bind the polymer to the substrate. In order to achieve this, the electrodes were immersed in 0.01M hydrochloric acid (HCl) for 15 minutes to initialise the SiO₂ substrate. They were then removed, rinsed with DI water and allowed to dry. After this, the electrodes were immersed in a 0.02M solution of 3-(trimethoxysilyl)propyl methacrylate for 1 hour. After which they were removed, rinsed with DI water and allowed to dry. After performing this process, the methacrylate groups now bonded to the surface of the substrate will take part in the polymerisation reaction, covalently binding the polymer to the substrate surface.

### OCPC Deposition and Polymerisation

To deposit the OCPC on the Interdigitated Electrodes (IDE) and/or to polymerise the resultant mixture, a small volume of the OCPC solution, between 2 and 5 μL is pipetted onto the IDE such that the solution completely covers the electrodes. The solution is then exposed to UV radiation for 60 seconds using a Dymax Bluewave 75 UV source. The resultant free-radical polymerisation will result in the OCPC covering the IDE.

The OCPC deposition was carried out manually using a micropipette. This may, however, readily be automated using robotic syringe dispensation techniques, capable of smaller (for example nanolitre (nL)) depositions. Liquid or polymer-based depositing techniques could also be used. These techniques include, but are not limited to, ink-jet printing, screen printing, and roll-to-roll printing.

Parameters such as electrode size, spacing, pattern and orientation can be varied to optimise electrode properties. This may improve electrical measurements, for example through the improvement of signal to noise ratios. Specific examples of the types of IDE modification envisaged include:
Reduction in the size of the IDEs to allow for smaller composite droplets to be used (allowing for faster response times and/or improving electrical signal to noise ratios).
Creation of patterned arrays of IDEs, for multiple sensors on a single chip.
Modification of the chip design to include passivation layers, more complex geometry or on-chip microfluidic delivery.
Alternative low-cost and disposable materials for chip fabrication, e.g. paper, plastic.
Screen-printed electrodes (e.g. carbon), roll-to-roll and/or ink-jet printing of conductive inks.

### Polymers

The base polymer used was a polyacrylamide hydrogel, consisting of a monomer (acrylamide, 15% by weight) and a standard cross-linker (N,N′-methylene-bisacrylamide) with a concentration that can be varied to tune the physical characteristics of the resultant hydrogel as required (typically ranging between 0.2 and 2.0 mol % w.r.t. monomer). To this a photo-initiator (1-hydroxycyclohexylphenylketone) and sodium chloride (NaCl, 150 mM) were added. The photo-initiator serves to enable the polymerisation to be initiated by UV radiation and the NaCl will stabilise any oligonucleotides incorporated into the polymer. The solvent used for the hydrogels can be an equal mixture of dimethyl sulfoxide (DMSO) and 1 mM phosphate buffer. The exact method for preparing these hydrogels varies according to the conductive component used (this method was adapted from [1]). In additional experimental work, it has been found that DMSO can be omitted from the methodology without adverse effects, and indeed omission of DMSO has become the standard approach (FIG. 13 shows a graph of an experiment wherein the method described herein was repeated, but without the use of DMSO). However, in some cases a solvent to act as an aid to dispersion may be beneficial.

The oligonucleotide cross-linked hydrogel can, however, be constituted from any hydrophilic polymer including, but not limited to, poly(N-isopropylacrylamide)s; poly(vinyl alcohol)s; poly(ethylene glycol)s; poly(N,N′-dialkyl acrylamide)s, poly(2-hydroxypropyl (meth)acrylate)s, poly(2-methoxyethyl acrylate)s and poly(di(ethylene glycol)methyl ether methacrylate)s.

In each instance, the hydrogel could be further tuned by copolymerisation of the main monomer with a secondary monomer. This monomer does not necessarily need to be hydrophilic and can include the monomers forming the aforementioned polymers but also any other acrylate, methacrylate, vinyl acetate, styrene, acrylonitrile and other such olefin-containing monomers. The composition of the material will usually contain a smaller fraction of this secondary monomer (<10%).

### Conductive Fillers

When the percolating composite comprises carbon nanopowder, this pre-gel mixture is then added to the desired mass of carbon nanopowder, and the nanopowder is dispersed within the solution by high power sonication using a 500 W sonic probe set at 60% amplitude in pulsed mode (10 seconds on, 15 seconds off) for 12 minutes 15 seconds. This sonication takes place indirectly, meaning that the sonicator is used to sonicate a bath of water and the composite mixture is placed in that bath in close proximity to the probe tip. In another example that has been successfully carried out, the sample was suspended in a low power sonicating water bath. In yet another successful example, the samples were simply agitated by hand for 5 minutes.

When the percolating composite comprises carbon nanotubes, the carbon nanotubes are suitably multi-walled 10 nm×6 μm. The desired weight, typically 2 mg/ml, and potentially less, of nanotubes is dispersed in an equal mixture of DMSO and 1 mM phosphate buffer by low power sonication. The mixture is sonicated in a sonic bath (ca. 12 W) for 8-24 hours. To aid dispersion a surfactant (sodium dodecylbenzenesulfonate) was added at ten times the weight of carbon (method adapted from reference [2]). After sonication, the nanotube suspension was added to a dry mixture of the required masses of the monomer, cross-linker, photo-initiator and NaCl to make a pre-gel/nanotube suspension solution of the desired concentration.

The academic literature provides numerous examples of uses of other conductive filler powders, combined with insulating polymers, to make percolating composites with the similar property of electrical resistance change with volume change (i.e. piezoresistive composites). Commonly referenced examples, which could easily be incorporated (if desired), include carbon black, nickel, gold, silver, zinc, copper, etc. Highly conductive fillers are preferable, to maximise the resistance change upon swelling. Most of the literature has previously focussed upon micro-sized powders, for their low cost and ease of purchase. However, for future low-cost sensors nano-sized powders are preferred for their greater compatibility with large-scale printing processes, such as ink-jet printing. Smaller filler particles also permit the further miniaturisation of sensor volumes (for faster response), whilst maintaining uniform particle distribution and good associated electrical repeatability.

As an alternative to metallic filler particles, one may also use intrinsically conductive polymers, which could be blended with the aforementioned insulating (sensing) polymer materials. Examples of conductive polymer fillers include, but are not restricted to: poly(thiophene) (PT), poly(3,4-ethylenedioxythiophene) (PEDOT), poly(p-phenylene sulphide) (PPS), poly(aniline)s (PANI), poly(pyrrole)s (PPY), poly(acetylene)s (PAC), poly(p-phenylene vinylene) (PPV), and mixtures and blends thereof.

In the instance whereby alternating current (AC) electrical impedance measurements are utilised for electrical measurement of response (see below for more details), the filler particles could be used to tune the dielectric properties of the composite (and hence both its resistance and complex impedance) to optimise the measurable response to swelling. In this instance, it may be preferable to lower or raise the impedance of the composite to a region with greatest sensitivity. In these instances, other conductivities of filler particles may be preferable, and may include semiconductor powders, or even insulating particles.

### Oligonucleotide Cross-Linkers

The hydrogel was functionalised by the addition of a frangible, oligonucleotide-based cross-linker. These cross-linkers consist of two oligonucleotide sequences, each terminated at their 5′ ends with a polymerisable chemical modifier, Acrydite (Error! Reference source not found.4), an acrylamide derivative which participates in the polymerisation reaction, thereby anchoring the oligonucleotides to the hydrogel network. The oligonucleotides were designed so as to be partially complementary to each other so that they will hybridise and form a cross-link, and provided with an acrydite molecule at their ends (FIG. 4). Upon the introduction of a nucleotide strand that is perfectly complementary to one of the two cross-linking strands, this strand will preferentially hybridise with its matching strand, thus breaking the cross-link. This process results in a change in cross-link density which will affect the swelling characteristics of the hydrogel. This change in swelling can be transduced by analysis of the electrical properties of the resultant composites.

All functionalised oligonucleotide sequences for use in the cross-linkers and as target and control sequences were sourced from Integrated DNA Technologies. The oligonucleotide target was designed so as to be a DNA analogue of the miRNA mir-92a. This was chosen for its association as a confirmed biomarker for leukaemia, although any sequence or miRNA could be chosen. The probe strand (also referred to as a “sensor strand” was designed to be exactly complementary to the target sequence. Using a custom MatLab algorithm, the blocker sequence was designed such that the first ten bases (from the 5′ end) had a random sequence and the remaining twelve bases were perfectly complimentary to the final twelve bases of the sensor strand, as shown in FIG. 4. A random sequence was designed to act as a control. The sequences of all of the nucleic acid strands described above are shown below in Table 1. “Acr” denotes the Acrydite moiety.

TABLE 1

| Strand | Sequence (5′-3′) |
|---|---|
| Sensor | Acr-ACAGGCCGGGACAAGTGCAATA |
| | (SEQ ID NO 1) |
| Blocker | Acr-TAGTGCGTTTTATTGCACTTGT |
| | (SEQ ID NO 2) |
| Target | TATTGCACTTGTCCCGGCCTGT |
| | (SEQ ID NO 3) |
| Random control | ACGTCTAGACGTAACGAAGGTC |
| | (SEQ ID NO 4) |

To incorporate the oligonucleotide cross-linkers into the polymer composite the probe and blocker functionalised oligonucleotides were mixed together in 1 mM phosphate buffer at the desired concentration (and suitably heated to 95° C. for 2 minutes then cooled to room temperature) before being extracted from the solution using a standard ethanol precipitation, leaving dried functionalised oligonucleotides. To this, the appropriate volume of the pre-gel solution/nanoparticle suspension was added and left for approximately three hours to allow the oligonucleotides to fully dissolve and hybridise. (In the case of carbon nanopowder the resultant mixture was shaken or sonicated using the same method as described before in order to re-suspend the nanopowder, which will typically have fallen out of suspension during the three hours.)

The final composite hydrogel is made by a radical polymerisation initiated by a light induced in situ generated radical. This radical polymerisation may also be initiated by thermal or light induced radical formation from peroxides, metal-alkyl, azo or other related compounds. Alternatively, the hydrogel can be formed by other strategies including coordination-insertion, anionic, cationic, controlled radical, ring-opening and other polymerisation techniques depending upon the monomer and end-functionality chosen.

FIG. 4 shows the swelling response of an oligonucleotide cross-linked hydrogel upon introduction of the target nucleic acid. As can be seen, the probe (sensing) strand 472 and a blocking strand 474 form a partially complementary oligonucleotide cross-link. Upon the introduction of the target nucleic acid 476, the cross-link is cleaved as the target nucleic acid 476 hybridises preferentially with the sensing strand 472. This causes the cross-link density of the polymer to become reduced, thus allowing the polymer 478 to swell 480. Also shown is the chemical modifier acrydite 482 to which the nucleic acid sequences of the cross-linker are joined.

### Electrical Measurements

DC resistance measurements were performed using a Keithley 2000 multimeter in 2-terminal resistance mode to measure the resistance of the OCPCs on the IDEs. This technique was used to measure the changes in resistance that occurred as the OCPCs moved between the dried state (in ambient room conditions) and the saturated state (at equilibrium in a phosphate buffer solution). Sensor response can be measured in a number of ways. Comparisons can be made between the saturated state with and without the presence of the target oligonucleotide (i.e. where the biosensor is pre-swollen) or between the dried state and the saturated state. The choice between these methods will depend on sensor performance and environmental aspects of practical usage.

A custom-made current source (FIG. 5) was also used to supply a constant current of 1.11 pA and a multimeter was used to measure the DC voltage. FIG. 5 shows a circuit diagram 590 of the current source. R_{C} is a 5.1MΩ resistor, R_{L} is the OCPC electrode under test. The Operational Amplifiers are LM 741CNs. The resultant output current was determined via calibration to be 1.11 pA. The resistance was inferred from these two pieces of information. Low currents are chosen to minimise unwanted effects such as Joule heating or space-charge limited currents, which can cause drift in the sensor's response. A constant current is also preferable, so as to avoid resistance measurement inaccuracies caused by the non-linear current-voltage characteristics (non-ohmic behaviour) of these types of composites.

### Alternative/Future Electrical Measurement Techniques:

AC impedance measurements: Investigations are currently underway to determine if AC measurements of complex electrical impedance (i.e. capacitance and/or inductance) offer benefits for measuring sensor response compared to DC resistance measurements. The technique is hypothesised to eliminate contributions from competing ionic conduction mechanisms.
AC impedance spectroscopy methods (or self-resonant frequency (SRF) measurements) are well documented and could also be used. A complex equivalent circuit is formed through the capacitive junction of the electrodes with the polymer (and also with the conductive particles distributed within it). The resonant frequency of this circuit will change upon polymer swelling, and can be used as a sensitive detection method.
Pattern recognition software and techniques such as Principle Component Analysis (PCA) have been used extensively in electronic nose technologies, to help provide highly specific recognition of complex aromas. A similar approach is also envisaged in the present invention, whereby multiple miRNAs are detected on a single disposable chip using multiple OCPC sensors, each targeting a different oligonucleotide sequence. Detection and diagnosis of the disease state will be a complex combination of multiple biomarkers, quite possibly at different concentrations. The user of the device will not necessarily have the knowledge or skills to interpret the data of individual miRNA signals from individual sensors, and so pattern recognition will be used to convert raw data into a useful diagnosis. As future medical research advances are made, the algorithms and libraries for comparing and recognising biomarker patterns will be updated to the device, enabling it to continually improve its performance.
On-chip integration: The above electrical and software techniques could all feasibly be integrated on-chip with the OCPC sensors themselves, using advanced (e.g. CMOS) microelectronic fabrication techniques, and thus could be integrated into a wide range of electrical diagnostic devices. However, due to the disposable and non-reversible response of the OCPC, it is perhaps more likely that a separate interrogation module is developed, containing the electrical measurement circuitry, and into which the disposable sensor chip is inserted.

### Sensing and Other Results

FIG. 6 shows the percolation curve for carbon nanopowder in polyacrylamide with a bisacrylamide cross-linking density of 0.8 mol % (without any oligonucleotide cross-linkers). It shows how the conductivity of the OCPC varies with nanoparticle concentration and the concentration at which percolation occurs. Just above the percolation threshold, the composite will be the most sensitive to any changes in volume that occur from swelling. These measurements were taken when the composite was dried in air at ambient conditions and were taken using the multimeter in 2-terminal resistance mode. The sharp change in conductance at a critical volumetric loading of carbon powder provides an enhanced electrical transduction to polymer swelling.

FIG. 7 show how the resistance of OCPCs change over time upon being immersed in 10 μM oligonucleotide solutions. The data sets represented with a circle and a triangle (instance 1 and instance 2) show separate repetitions of the resistance change of OCPCs immersed in a solution containing the complementary analyte strand. The dataset represented with a square show the resistance of an OCPC immersed in a control solution containing a random oligonucleotide strand. It can be seen that there is a clear difference in the swelling behaviour of the OCPCs in the analyte solution as compared to the control solution. In particular, we notice that there is a difference in the rate of swelling response between the positive and negative instances, which could be used to provide discrimination. One could simply measure the time taken to reach a pre-determined swelling amount (resistance value), or compare other temporal characteristics between OCPC responses. Alternatively, one could simply measure resistance after a specified time (in this case approximately 200 s) and compare the value of resistances at this point in time. In some embodiments of the sensor, one may also be able to compare differences in the final saturated and fully swollen state between various OCPCs, although this particular example shows the final swollen state to be similar in all 3 instances.

These results clearly demonstrate that a biosensor according to the present invention can successfully detect an oligonucleotide in a sample. Of course, the biosensor will likely benefit from further optimisation, but the utility and promise of the present invention has been clearly demonstrated.

Integration of an Aptamer Sequence into a Polymer Matrix

A nucleic acid aptamer can be introduced using an analogous method to that described above for miRNA-targeting nucleic acid probes.

Aptamer sequences are available from a variety of commercial sources, and are widely described in the literature. For example, Base Pair Biotechnologies, Inc. sell validated aptamers for protein, cell-surface, peptide, and small molecule targets (see http://www.basepairbio.com/project-phases-2/existing-aptamers/)

For the present example we will refer to an aptamer probe targeting ampicillin, but aptamers for other targets can be readily used.

A commercially available ampicillin aptamer, Known as Ampicillin Aptamer 5E01#284 is available from Base Pair Biotechnologies, Inc. (see http://www.basepairbio.com/wp-content/uploads/2013/04/Ampicillin-ATW0001-284-datasheet.pdp. Several other aptamer sequences for ampicillin are described in the literature, including 5′-CACGGCATGGTGGGCGTCGTG-3′ (SEQ ID NO 5), 5′-GCGGGCGGTTGTATAGCGG-3′ (SEQ ID NO 6), and 5′-TTAGTTGGGGTTCAGTTGG-3′ (SEQ ID NO 7).

To combine the aptamer sequence with a polymerisable monomer a primary amine functionalized ampicillin aptamer sequence, e.g. NH₂(5′-CACGGCATGGTGGGCGTCGTG-3′), NH₂(5′-GCGGGCGGTTGTATAGCGG-3′) or NH₂(5′-TTAGTTGGGGTTCAGTTGG-3′) is reacted with acryloyl chloride or bromide to afford the acrylamide terminated oligonucleotide sequence (i.e. CH2═CHN(═O)— sequence). This gives a monomer which is essentially equivalent to the acrydite modified sequences discussed above. In some cases a spacer sequence (e.g. from 1 to 20 nucleotides in length) can be provided between the acrylamide and the aptamer sequence.

The sequence is incorporated into the swellable polymer along with a poorly matched blocking strand to form frangible cross-links using the methodology described above. Binding of the target (i.e. in this case ampicillin) cleaves the cross-link, permitting increased hydrogel swelling.

In an alternative approach, two blocker oligonucleotide strands which are adapted to hybridise with the aptamer sequence are integrated into the polymer, using the methodology described earlier. The aptamer sequence (now not functionalised) is added to the polymer and hybridises to and bridges these two blocker stands forming partial matches (e.g. of ˜10 base pairs with each side). The presence of the target compound (i.e. ampicillin) results in dissociation of the aptamer sequence from the three part cross-link permitting increased swelling and a transduced response.

In an alternative approach, ampicillin is integrated into the polymer by functionalisation of the amine (NH₂) of the ampicillin in an analogous way to functionalisation of the oligonucleotides discussed above. Optionally a short spacer chain is used to space the ampicillin from the polymer backbone (e.g. a spacer of 10 of fewer carbons in length, giving (CH₂)ₙNHCOCH═CH₂— where n=0 to 10).

Further details of the integration of aptamers into hydrogels are described in Yang et al. [29].

Modifications and improvements can be made without departing from the scope of the invention. For example, alternative transduction mechanisms are also possible. These include a variety of microelectromechanical systems (MEMS)-based approaches, which could be combined with the polymers.

### FURTHER EXEMPLIFICATION

### Example 1—Additional Polymers Using Acrylamide and N-isopropylacrylamide Monomers

Further polymer variants were produced using varying amounts of acrylamide monomers, and using a combination of acrylamide and N-isopropylacrylamide monomers. The methodology used was generally as described above, except where indicated otherwise.

All samples were prepared in pH 7.4 phosphate buffer (1 mM) with a NaCl concentration of 150 mM unless otherwise stated. Monomer/pre-gelator solutions were prepared from stock solutions of acrylamide (AAm); and N-isopropylacrylamide for copolymer gels, N,N′-methylene bisacrylamide (Bis-AAm) and 1-hydroxycyclohexyl phenyl ketone (in ethylene glycol). Mixing of these stocks gave final concentrations of 10 or 15 wt % total monomer, 0.6 mol % crosslinker and 0.13 mol % initiator, respectively. The stock solution was then pipetted into a 1.5 mL Eppendorf centrifuge tube containing DNA to give a final oligonucleotide concentration of 0.4 mol %.

FIG. 8 shows the selective swelling of the polymer and copolymer gels (10 wt %) in the presence of the analyte strand (SEQ. ID: 3) compared to the random control strand (SEQ. ID: 4). A larger increase in swelling due to cleavage of the oligonucleotide crosslinks by the analyte strand is observed. A slightly lower overall swelling is observed for 50:50 copolymer gels due to the decreased hydrophilicity of N-isopropylacrylamide.

The following variations were produced:
1. AAm gels at 10, 15, 20 or 25 wt %
   Bis 0.4, 0.6, 0.8, 1.0 and 1.2 mol % wrt AAM
   PDA (Piperazine di-acrylamide) use on a gram for gram basis instead of Bis
2. AAm gels with DNA at 10 or 15 wt %
   Bis 0-1 mol % wrt AAM
   DNA 0.1-2 mol % wrt AAM
   Typically 0.6 mol % Bis, 0.4 mol % DNA wrt AAM
3. 50:50 AAm/NIPAAm gels at 10 and 15 wt %, and 80:20 and 20:80 AAm/NIPAAm gels (15 wt %). All samples prepared with and without DNA crosslinks (0.6 mol % Bis with or without 0.4 mol % DNA)

All of these variants were shown to perform satisfactorily, in terms of their intended purpose, though the individual properties of each variant was of course different, and would be suited to differing uses.

### Example 2—Aptamer-Based Frangible Crosslinkers

Polymers were produced using aptamer-based cross linkers, using approaches based upon Yang et al. [29]. In this example two different approaches of using an aptamer that selectively bind adenosine were used. The two approaches are shown schematically in FIG. 9, and are described below. The aptamer has the sequence 5′-ACCTGGGGGAGTATTGCGGAGGAAGGT-3′ (SEQ ID NO: 8).

### Approach 1:

In Approach 1, two nucleic acid strands were linked to the polymer (referred to as Blocking Strands 1 and 2), and a ‘Sensing’ nucleic acid strand comprising the aptamer sequence was provided as a third sequence adapted to bind to both the blocking strands. Thus, the two blocking strands combine with the sensing strand to form a frangible cross-linker.
The aptamer containing Sensing Strand has the sequence

(SEQ ID NO: 9)
5′
ACTCATCTGTGAAGAGAACCTGGGGGAGTATTGCGGAGGAAGGT-3′

—the actual aptamer sequence is underlined.
Blocking Strand 1 has the sequence 5′-TGAGTAGACACT-3′ (SEQ ID NO: 10), which is complementary to the 5′ end of the Sensing Strand.
Blocking Strand 2 has the sequence 5′-TCTCTTGGACCC-3′ (SEQ ID NO: 11), which is complementary to the Sensing Strand in the region immediately 3′ of the region bound by Blocking Strand 1 and the first 7 bases of the 5′ end of the aptamer sequence. It is Blocking Strand 2 that actually blocks the active region of the aptamer sequence.

As can be seen in the FIG. 9, when adenosine is introduced in a sample, the Sensing Strand binds to adenosine and is released from Blocking Strand 2, thus cleaving the frangible cross-linker. The sensing strand remains bound to Blocking Strand 1. The polymer is thus able to swell in the presence of the sample, thus resulting in a detectable signal.

Dried oligonucleotide crosslinks were prepared as described previously, using isopropanol rather than ethanol. AAm (10 and 15 wt %) gels with aptamer crosslinks (0.6 mol % bis and 0.4 mol % aptamer wrt AAm) were prepared from the stock solutions and methods described for AAm gels above with 300 mM NaCl rather than 150 mM. Selective swelling was obtained in response to 2 mM adenosine solution compared to buffer.

### Approach 2:

In Approach 2 both the sensing aptamer containing strand and the corresponding blocking strand are linked to the polymer. Thus, there is no third strand in Approach 2, and instead the Blocking and Sensing Strands together form the frangible cross-linker.
The aptamer containing ‘sensing’ strand has the sequence

(SEQ ID NO: 12)
5′
AGAGAACCTGGGGGAGTATTGCGGAGGAAGGT-3′

—the actual aptamer sequence is underlined.
The Blocking Strand has the sequence 5′-TCTCTTGGACCG-3′ (SEQ ID NO: 11), which is complementary to region extending from the 5′ end of the sensing strand to the seventh base of the aptamer strand.

As can be seen in the FIG. 9, when adenosine is introduced in a sample, the Sensing Strand binds to two molecules of adenosine and is released from Blocking Strand, thus cleaving the frangible cross-linker. The polymer is able to swell in the presence of the sample, thus resulting in a detectable signal.

Dried oligonucleotide crosslinks were prepared as described previously, using isopropanol rather than ethanol. AAm (10 and 15 wt %) gels with aptamer crosslinks (0.6 mol % bis and 0.4 mol % aptamer wrt AAm) were prepared from the stock solutions and methods described for AAm gels above with 300 mM NaCl rather than 150 mM. Selective swelling was obtained in response to 2 mM adenosine solution compared to buffer (FIG. 10). Swelling volumes were lower than the previous aptamer crosslinked gels.

### Example 3—Morpholino Frangible Cross-Linkers

Morpholinos were functionalised for incorporation into the polymer framework as shown in the reaction below.

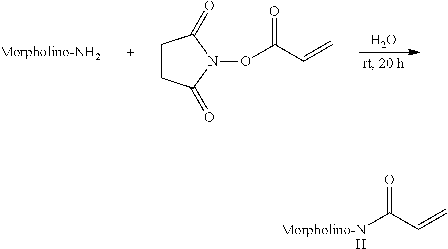

‘Sensor/block’ morpholino oligonucleotide material (with an ethyl linker between the primary amine and oligonucleotide sequence), with the same sequences as SEQ ID NO: 1 and 2, respectively, but with the Acrydite group replaced with a primary amine, was dissolved in distilled water (at a concentration of 1 mM) in a round bottom flask and 2 molar equivalents of an aqueous solution of N-succinimidyl acrylate was added. This was left to stir at room temperature for ˜20 hrs. MALDI-ToF spectroscopy was carried out to determine product formation. After lyopholisation, the product was washed with excess hot (˜50° C.) ethyl acetate to remove succinimide and then freeze-dried to afford the acrylamide-functionalised product.

All gel samples were prepared from the stock solutions and methods described for polymer and copolymer gels above, but the monomer/pre-gelator solution was then added to an Eppendorf tube containing the modified morpholino sensor and blocker strands to give a final morpholino crosslink concentration of 0.4 mol % and a 0.6 mol % bis concentration.

AAm and 50:50 (10 and 15 wt %) gels with morpholino crosslinks (0.6 mol % bis and 0.4 mol % morpholino) can be formed and display selective swelling between immersion in a solution containing analyte (SEQ ID NO:3) and the random control (SEQ ID NO:4). Overall swelling is less in gels containing morpholino crosslinks compared to those with DNA crosslinks. Selective swelling was displayed and overall swelling was also lower than DNA gels when a morpholino material with a pentyl linker, as opposed to an ethyl linker, (between oligonucleotide sequence and acrylamide end of the molecule) was used. The swelling was slightly higher than for morpholino crosslinked gels containing the ethyl linker moiety (for AAm (10 wt %) gels); shown in FIG. 11. This pentyl linker-containing morpholino material was synthesised by GeneTools, which was then purified using HPLC.

Gels (10 wt % AAm) containing morpholino crosslinks (ethyl linker) or a morpholino (with a pentyl linker between acrylamide moiety and oligonucleotide sequence) sensor strand and a DNA blocker strand (‘hybrid’ gel) seem to be less effected by salt concentration (NaCl concentration from 0-200 mM) compared to a purely DNA-containing gel (FIG. 12). Samples were prepared as before, but with solutions containing NaCl concentrations ranging from 0-200 mM and were only swollen when the target analyte was present (SEQ. ID NO: 3).

### Example 4—Alternative Initiators

A variety of initiators can be used to initiate polymerisation. Initiation using ammonium persulfate (APS) was used further example, as follows:

Pre-gelator solutions of AAm (10 and 15 wt %) gels with no oligonucleotide crosslinks (0.6 mol % bis wrt AAm) were prepared without photoinitiator. Under inert atmosphere, in this case nitrogen, APS (Ammonium Persulfate) 0.125 mol % wrt AAM, and TEMED (tetramethylethylenediamine) 0.5 or 1.0% final concentration were added to the pre-gelator solutions. The appropriate volume (1 or 2 μL) was then pipetted on a silanised silicon surface described previously and allowed to polymerise in an inert atmosphere for 90-120 minutes. For oligonucleotide crosslinked gels add the APS and TEMED after solubilising the dried oligonucleotides in the pre-gelator solution.

### REFERENCES

1. S. Tierney, D. R. Hjelme, B. T. Stokke, Analytical Chemistry, 80, 5086 (2008)
2. M. F. Islam, E. Rojas, et al., Nano Letters, 3, 269 (2003)
3. M. Gao, K. Gawel, B. T. Stokke, Soft Matter, 7, 1741 (2011)
4. M. Gao, K. Gawel, B. T. Stokke, Soft Matter, 7, 1741 (2011)
5. K. Gawal, D. Barriet, et al., Sensors, 10, 4381 (2010)
6. K. Gawal, B. T. Stokke, Soft Matter, 7, 4615 (2011)
7. S. Tierney, B. T. Stokke, Biomacromolecules, 10, 1619 (2009)
8. M. C. Burl, B. J. Doleman, et al., Sensors and Actuators B, 72, 149 (2001)
9. B. J. Doleman, N. S. Lewis, Sensors and Actuators B, 72, 41 (2001)
10. B. J. Doleman, M. C. Longergan, et al., Analytical Chemistry, 70, 4177 (1998)
11. B. J. Doleman, R. D. Sanner, et al., Analytical Chemistry, 70, 2560 (1998)
12. B. J. Doleman, E. J. Severin, N. S. Lewis, Proc Natl Acad Sci USA, 95, 5442 (1998)
13. M. C. Lonergan, E. J. Severin, et al., Chemical Materials, 8, 2298 (1996)
14. E. J. Severin, B. J. Doleman, N. S. Lewis, Analytical Chemistry, 72, 658 (2000)
15. E. J. Severin, R. D. Sanner, et al., Analytical Chemistry, 70, 1440 (1998)
16. P. J. W. Hands, P. J. Laughlin, D. Bloor, Sensors and Actuators B: Chemical, 162, 400 (2012)
17. D. C. Ferrier, M. P. Shaver, P. J. W. Hands, Biosensors and Bioelectronics, 68, 798 (2015)
18. M. Lee, C.-Y. Chen, et al., Advanced Materials, 24, 1759 (2012)
19. L. Cui, Conducting polymer based QCM-interdigitated electrode hybrid electronic nose-system. 1999, University of Glasgow: Glasgow.
20. L. Cui, M. J. Swann, et al., Sensors and Actuators B, 66, 94 (2000)
21. O. B. Ayyub, J. W. Sekowski, et al., Biosensors and Bioelectronics, 28, 349 (2011)
22. P. Jiang, D. W. Smith Jr., et al., Advanced Materials, 17, 179 (2005)
23. Y. Kang, J. J. Walish, et al., Nature Materials, 6, 957 (2007)
24. W. Lee, J. Yoon, et al., Macromolecules, (2013)
25. C. J. Musto, K. S. Suslick, Current Opinion in Chemical Biology, 14, 758 (2010)
26. N. A. Rakow, A. Sen, K. S. Suslick. A colorimetric nose: “Smell-seeing”. in ISOEN8. 2001. Washington D.C.: ECS.
27. H. Wang, K.-Q. Zhang, Sensors, 13, 4192 (2013)
28. M. C. Chiappelli, R. C. Hayward, Advanced Materials, 24, 6100 (2012)
29. Yang et al. Engineering Target-Responsive Hydrogels Based on Aptamer-Target Interactions; J Am Chem Soc. 2008 May 21; 130(20): 6320-6321. doi:10.1021/ja801339w

1. A biosensor comprising:
a swellable biologically sensitive element comprising a nucleic acid probe; and
a physicoelectrical transducer associated with the swellable biologically sensitive element.

2. The biosensor of claim 1 wherein the target for the probe is a nucleic acid, a protein/peptide, drug, lipid, polysaccharide, other small molecules, or a cell surface.

3. The biosensor of claim 1 wherein the nucleic acid probe is an aptamer.

4. The biosensor of any claim 1 wherein the physicoelectrical transducer is adapted to transform the physical effect of swelling of the swellable biologically sensitive element into a detectable/measurable electrical indicator.

5. The biosensor of claim 4 wherein the electrical indicator to be detected is a change in electrical impedance or admittance, such as resistance, conductance, reactance, susceptance, capacitance, inductance, or combinations thereof.

6. The biosensor of any claim 1 wherein the nucleic acid probe defines part of a frangible cross-linker in a swellable polymeric material, wherein the cross-linker is cleaved when a target binds to the nucleic acid probe.

7. The biosensor of any claim 1 wherein the swellable biologically sensitive element comprises at least one pair of at least partially complementary nucleic acid strands, at least one strand of each pair comprising a nucleic acid probe.

8. The biosensor of claim 6 wherein the frangible cross-linker comprises at least one pair of partially complementary nucleic acids which comprise a nucleic acid probe strand and a blocker strand.

9. The biosensor of claim 6, wherein the nucleic acid probe is adapted to bind to a nucleic acid target through hybridisation, or is an aptamer adapted to bind to a non-nucleic acid target.

10. (canceled)

11. The biosensor of claim 6, wherein the swellable biologically sensitive element comprises a frangible crosslinker comprising an aptamer probe and a corresponding target for the aptamer.

12. (canceled)

13. (canceled)

14. (canceled)

15. The biosensor of claim 1, wherein the nucleic acids probe comprises a nucleic acid analogue/mimetic or other form of modified or altered nucleic acid.

16. (canceled)

17. (canceled)

18. (canceled)

19. (canceled)

20. The biosensor of claim 1, comprising a swellable polymer which comprises non-frangible cross-linkers and frangible nucleic acid cross-linkers.

21. The biosensor of claim 1, wherein the swellable biologically sensitive element comprises a hydrophilic, water-swellable polymer.

22. (canceled)

23. (canceled)

24. (canceled)

25. (canceled)

26. (canceled)

27. The biosensor of claim 1, wherein the swellable biologically sensitive element comprises an oligonucleotide cross-linked polymer composite (OCPC).

28. The biosensor of claim 1, wherein the physicoelectrical transducer comprises a piezoresistive material or electrically percolating composite material, capable of exhibiting changes in its electrical impedance due to externally-induced changes in its volume.

29. (canceled)

30. The biosensor of claim 28 wherein the percolating composite comprises conductive particles distributed in the swellable biologically sensitive element.

31. (canceled)

32. (canceled)

33. (canceled)

34. The biosensor of claim 30, which is adapted such that swelling of the polymer upon cleavage of the nucleic acid cross-linkers results in a change in volumetric fraction of the conductive particulate material within or across at least part of the percolation threshold.

35. The biosensor of claim 1, which comprises a substrate comprising electrodes to allow detection and/or measurement of the electrical signal or change in electrical properties of the biosensor as a result of swelling of the swellable biologically sensitive element.

36. (canceled)

37. (canceled)

38. (canceled)

39. The biosensor of claim 1 connected to a measurement apparatus adapted to detect and/or quantify an electrical indicator from the biosensor.

40. (canceled)

41. A method for detecting the presence or amount of a target in a sample, the method comprising:
a) providing a biosensor according to claim 1;
b) exposing the biosensor;
c) observing for an electrical signal or change in electrical property in the biosensor as a result of exposure to the sample;
d) optionally determining the magnitude of said change; and/or
e) optionally determining the speed of said change; and
f) determining therefrom the presence and/or amount of said target in said sample.

42. (canceled)

43. (canceled)

44. (canceled)

45. A method of producing a biosensor, the method comprising:
a) preparing a swellable biologically sensitive element comprising a nucleic acid probe; and
b) associating a physicoelectrical transducer with the swellable biologically sensitive element.

46. The method of claim 45 comprising:
providing suitable monomers to form a swellable polymer matrix,
providing nucleic acid cross-linking monomers,
providing electrically conductive particles,
optionally providing non-frangible cross-linkers,
mixing the aforementioned together, and
causing the monomers, and optionally non-frangible cross-linkers to polymerise, thereby forming a swellable polymer comprising frangible nucleic acid cross-links having an associated a physicoelectrical transducer.


SEQUENCE LISTING (12 sequences)

SEQ ID NO: 1 - DNA, 22 bp, Artificial Sequence
  Sensor strand
acaggccgggacaagtgcaata

SEQ ID NO: 2 - DNA, 22 bp, Artificial Sequence
  Blocker strand
tagtgcgttttattgcacttgt

SEQ ID NO: 3 - DNA, 22 bp, Artificial Sequence
  Target strand
tattgcacttgtcccggcctgt

SEQ ID NO: 4 - DNA, 22 bp, Artificial Sequence
  Control strand
acgtctagacgtaacgaaggtc

SEQ ID NO: 5 - DNA, 21 bp, Artificial Sequence
  Aptamer binding to ampicillin
cacggcatggtgggcgtcgtg

SEQ ID NO: 6 - DNA, 19 bp, Artificial Sequence
  Aptamer binding to ampicillin
gcgggcggttgtatagcgg

SEQ ID NO: 7 - DNA, 19 bp, Artificial Sequence
  Aptamer binding to ampicillin
ttagttggggttcagttgg

SEQ ID NO: 8 - DNA, 27 bp, Artificial Sequence
  Aptamer binding to adenosine
acctgggggagtattgcggaggaaggt

SEQ ID NO: 9 - DNA, 44 bp, Artificial Sequence
  Sensing strand for binding adenosine
actcatctgtgaagagaacctgggggagtattgcggaggaaggt

SEQ ID NO: 10 - DNA, 12 bp, Artificial Sequence
  Blocking Strand 1
tgagtagacact

SEQ ID NO: 11 - DNA, 12 bp, Artificial Sequence
  Blocking Strand 2
tctcttggaccc

SEQ ID NO: 12 - DNA, 32 bp, Artificial Sequence
  Sensing strand for binding adenosine
agagaacctgggggagtattgcggaggaaggt